SEQUENCE LISTING (30 sequences)

SEQ ID NO: 1 - DNA, 22 bp, Artificial sequence
  source: /note=“Description of Artificial Sequence Synthetic polynucleotide”
gctgttctcgttcctcactgcc

SEQ ID NO: 2 - DNA, 38 bp, Artificial sequence
  source: /note=“Description of Artificial Sequence Synthetic polynucleotide”
tgaaaatgcgagtgtccataccctacccgcgttcggac

SEQ ID NO: 3 - DNA, 22 bp, Artificial sequence
  source: /note=“Description of Artificial Sequence Synthetic polynucleotide”
cgccaaatacgccttagcagac

SEQ ID NO: 4 - DNA, 36 bp, Artificial sequence
  source: /note=“Description of Artificial Sequence Synthetic polynucleotide”
tgaaaatggaagtgtcaggttcttcccgcgaaatcg

SEQ ID NO: 5 - DNA, 24 bp, Artificial sequence
  source: /note=“Description of Artificial Sequence Synthetic polynucleotide”
ccttcaattgcttggcggactcgg

SEQ ID NO: 6 - DNA, 40 bp, Artificial sequence
  source: /note=“Description of Artificial Sequence Synthetic polynucleotide”
tgataatgcaagtctcacaagatgagcgagtgtaccgatg

SEQ ID NO: 7 - DNA, 24 bp, Artificial sequence
  source: /note=“Description of Artificial Sequence Synthetic polynucleotide”
gctgtaactgtggtttccatgacg

SEQ ID NO: 8 - DNA, 36 bp, Artificial sequence
  source: /note=“Description of Artificial Sequence Synthetic polynucleotide”
tgaaaatgcaagtgtccgtgtggcttacctgctgcc

SEQ ID NO: 9 - DNA, 25 bp, Artificial sequence
  source: /note=“Description of Artificial Sequence Synthetic polynucleotide”
agcggggtatgagctttcctgttac

SEQ ID NO: 10 - DNA, 39 bp, Artificial sequence
  source: /note=“Description of Artificial Sequence Synthetic polynucleotide”
tcaaaatgcaagtgtccagtcgggcgaaatctgtgtacc

SEQ ID NO: 11 - DNA, 25 bp, Artificial sequence
  source: /note=“Description of Artificial Sequence Synthetic polynucleotide”
gatatcggatcgcaacaagacctcg

SEQ ID NO: 12 - DNA, 42 bp, Artificial sequence
  source: /note=“Description of Artificial Sequence Synthetic polynucleotide”
tgaagatggaagtgtctccgttgcgtaatatgtcaaggatgc

SEQ ID NO: 13 - DNA, 22 bp, Artificial sequence
  source: /note=“Description of Artificial Sequence Synthetic polynucleotide”
ggtcaattcctgtcgcagtacc

SEQ ID NO: 14 - DNA, 41 bp, Artificial sequence
  source: /note=“Description of Artificial Sequence Synthetic polynucleotide”
catgtgcctacacctggtccaaacagccttaggtcttatgg

SEQ ID NO: 15 - DNA, 21 bp, Artificial sequence
  source: /note=“Description of Artificial Sequence Synthetic polynucleotide”
gtacgctaacactgcacgacg

SEQ ID NO: 16 - DNA, 43 bp, Artificial sequence
  source: /note=“Description of Artificial Sequence Synthetic polynucleotide”
catgtgcatacacctggtaacactgatgaacgtggtacaccag

SEQ ID NO: 17 - DNA, 23 bp, Artificial sequence
  source: /note=“Description of Artificial Sequence Synthetic polynucleotide”
gttgaccgcaaatagagccaagc

SEQ ID NO: 18 - DNA, 42 bp, Artificial sequence
  source: /note=“Description of Artificial Sequence Synthetic polynucleotide”
catgtgcctacacgaggtattagcgagaacgggaccatcatg

SEQ ID NO: 19 - DNA, 22 bp, Artificial sequence
  source: /note=“Description of Artificial Sequence Synthetic polynucleotide”
cagcaacaacgattgtttcgcc

SEQ ID NO: 20 - DNA, 38 bp, Artificial sequence
  source: /note=“Description of Artificial Sequence Synthetic polynucleotide”
catgtccatacacctgcttcctctttagctgctggaac

SEQ ID NO: 21 - DNA, 25 bp, Artificial sequence
  source: /note=“Description of Artificial Sequence Synthetic polynucleotide”
gcacacttaggtgatttacctgcat

SEQ ID NO: 22 - DNA, 39 bp, Artificial sequence
  source: /note=“Description of Artificial Sequence Synthetic polynucleotide”
catatccctacacctgccacccgtgtggatcataataga

SEQ ID NO: 23 - DNA, 16 bp, Artificial sequence
  source: /note=“Description of Artificial Sequence Synthetic polynucleotide”
catgtgcctacacctg

SEQ ID NO: 24 - DNA, 16 bp, Artificial sequence
  source: /note=“Description of Artificial Sequence Synthetic polynucleotide”
tgaaaatgcaagtgtc

SEQ ID NO: 25 - DNA, 18 bp, Artificial sequence
  source: /note=“Description of Artificial Sequence Synthetic polynucleotide”
gcactcttccagccttcc

SEQ ID NO: 26 - DNA, 36 bp, Artificial sequence
  source: /note=“Description of Artificial Sequence Synthetic polynucleotide”
tgaaaatggaagtgtcagcactgtgttggcgtacag

SEQ ID NO: 27 - DNA, 22 bp, Artificial sequence
  source: /note=“Description of Artificial Sequence Synthetic polynucleotide”
cagagctcagggtgacagagag

SEQ ID NO: 28 - DNA, 36 bp, Artificial sequence
  source: /note=“Description of Artificial Sequence Synthetic polynucleotide”
tgaaaatgcaagtctcccacgacaccaaccaccagg

SEQ ID NO: 29 - DNA, 24 bp, Artificial sequence
  source: /note=“Description of Artificial Sequence Synthetic polynucleotide”
tgctgaacgcatttactgtcacgg

SEQ ID NO: 30 - DNA, 40 bp, Artificial sequence
  source: /note=“Description of Artificial Sequence Synthetic polynucleotide”
tgaaaatgcaagtctcaccatagctgatcatgcagcggta

(19) United States
(12) Patent Application Publication
(10) Pub. No.: US 2022/0145284 A1
(43) Pub. Date: May 12, 2022

(54) METHOD OF DETECTING MULTIPLE TARGETS BASED ON SINGLE DETECTION PROBE USING TAG SEQUENCE SNP

(71) Applicant: SEASUN BIOMATERIALS, Daejeon (KR)

(72) Inventors: Si Seok LEE, Daejeon (KR); Eun Ju YANG, Daejeon (KR); Kyung Tak KIM, Daejeon (KR); Mee-Hyang JEON, Daejeon (KR); Hee Kyung PARK, Daejeon (KR)

(73) Assignee: SEASUN BIOMATERIALS, Daejeon (KR)

(21) Appl. No.: 17/421,716

(22) PCT Filed: Jan. 22, 2020
(86) PCT No.: PCT/KR2020/001123

§ 371 Date: Jul. 8, 2021

(30) Foreign Application Priority Data
Jan. 24, 2019 (KR) 10-2019-0009061

(51) Int. Cl. C12N 15/10; C12Q 1/6888; C12Q 1/6818; G01N 21/64

(52) U.S. Cl. CPC C12N 15/1065; C12N 15/1096; C12Q 1/6888; G01N 2021/6432; G01N 21/6428; C12Q 2600/16; C12Q 1/6818

(57) ABSTRACT

The present invention relates to a method of detecting multiple targets based on a single detection probe, and more particularly to a method of detecting multiple targets by amplifying each target with primers including an SNP-containing tag sequence, hybridizing the amplification products with a single detection probe capable of binding to the tag sequences and designed such that melting temperatures are different from each other, and analyzing melting curves. A method of detecting multiple targets according to the present invention enables the detection of multiple targets using a single probe, and thus is useful for detecting multiple targets because false positives are reduced and multiple targets are detectable with high sensitivity and at a rapid rate.

This is a U.S. national phase application under 35 U.S.C. § 371 of Patent Cooperation Treaty Application No. PCT/KR2020/001123, filed Jan. 22, 2020, which claims priority from Republic of Korea Patent Application Serial No. KR 10-2019-0009061, filed on Jan. 24, 2019, and which incorporates by reference those PCT and Republic of Korea applications in their entireties.

### SEQUENCE LISTING

The instant application contains a Sequence Listing which has been filed electronically in ASCII format and is hereby incorporated by reference in its entirety. Said ASCII copy, created on Jul. 8, 2021, is named PF-B2362_ST25.txt and is 5,936 bytes in size.

### BACKGROUND OF THE INVENTION

### Technical Field

The present invention relates to a method of detecting multiple targets based on a single detection probe, and more particularly to a method of detecting multiple targets by amplifying each target with primers including a tag sequence designed such that melting temperatures of hybridized reaction products of amplification products and a detection probe are different from each other, and then hybridizing the amplification products with a single detection probe that binds to all of the tag sequences and analyzing melting curves.

### Discussion of the Related Art

The field of diagnosis of specific nucleic acids is used to distinguish single-nucleotide polymorphisms (SNPs), detect and identify pathogenic bacteria or viruses, and diagnose genetic diseases. Thus, many methods for rapidly and accurately detecting specific nucleic acids have been proposed, and many related studies are currently being conducted (W. Shen et al., 2013, Biosen. and Bioele., 42:165-172.; M. L. Ermini et al., 2014, Biosen. and Bioele., 61:28-37.; K. Chang et al., 2015, Biosen. and Bioele., 66:297-307.).

Specifically, the most commonly used method for detecting a specific nucleic acid includes a method using polymerase chain reaction (PCR) and methods using real-time PCR and multiplex polymerase chain reaction (multiplex PCR).

PCR is advantageous in terms of being able to bind to template DNA and enabling accurate amplification only of a target region of a gene to be detected through design of a primer or probe to which a fluorescent material and a quencher are bound. However, only one nucleic acid can be amplified in one reaction, and thus, when the number of nucleic acids to be amplified is large, the same operation must be repeated, which is cumbersome.

Real-time PCR measures amplification products in real time, reduces cross-contamination, and enables more accurate quantitative analysis. As conventional patent documents related to real-time PCR, there are U.S. Pat. Nos. 5,210,015, 5,538,848, and 6,326,145.

Existing real-time PCR methods are advantageous in terms of a homogeneous assay method in which amplification and detection are performed simultaneously, but have a problem of multiplicity in that the number of target nucleic acid sequences that can be simultaneously detected is limited due to a limitation in the type of fluorescent reporter molecules, which is the biggest obstacle to realizing high throughput. Existing thermocyclers capable of detecting target nucleic acid sequences in real time enable simultaneous detection of a maximum of 5-plex, and thus the number of target nucleic acid sequences that can be detected simultaneously is limited, and a lot of time and additional expensive real-time monitoring equipment are required in order to analyze a sample having a large volume.

A TaqMan probe method (U.S. Pat. No. 5,210,015) and a self-quenching fluorescence probe method (U.S. Pat. No. 5,723,591), which are representative real-time PCR methods, have a problem of the occurrence of false positives due to non-specific binding of a dual-labeled probe, and thus it is practically difficult to perform 5-plex reactions, and specialized skills and know-how are necessarily required.

Since a conventional real-time PCR method simultaneously performs amplification and detection, there is a limitation in high throughput of real-time PCR equipment.

Multiplex PCR is advantageous in that several polymerase chain reactions are carried out in a single tube, thus simultaneously analyzing a plurality of nucleic acids. However, because many primers or probes are simultaneously used in one tube, cross reactions between the probes and the primers or between the primers occur, and thus the number of nucleic acids that can be amplified at one time is limited, a lot of effort and time is required to determine reaction conditions, and good results cannot be obtained in terms of sensitivity and specificity (Hardenbol et al., 2003, Nat. Biotechnol., 21:673.).

In addition, since one nucleic acid to be detected can be labeled with only one fluorescent material and equipment currently used for detecting fluorescent materials has a limitation in that the number of fluorescent channels that can be simultaneously analyzed at one time is limited to typically 4 types to 7 types, there is a problem in that two or more identical operations must be repeated in order to analyze 8 or more nucleic acids.

Therefore, recent studies have been actively conducted to enable mass analysis by simultaneously amplifying a plurality of nucleic acids using common primers without using multiplex polymerase chain reaction. Representative technologies include SNPlex, Goldengate assay, molecular inversion probes (MIPs), and the like.

SNPlex is a method which involves performing a purification process using an exonuclease after oligonucleotide ligation assay (OLA) and performing polymerase chain reaction amplification with common primer base sequences present at opposite ends of a probe, and then finally performing analysis in a DNA chip using a ZipCode base sequence included in the probe (Tobler et al., J. Biomol. Tech., 16:398, 2005).

The Goldengate assay is a method in which an allele-specific primer extension reaction is performed on genomic DNA immobilized on a solid surface using an upstream probe, after which DNA is ligated with a downstream probe and washed to remove probes not ligated to the DNA, the DNA is amplified using common primer nucleotide sequences included in the probes, like the case of SNPlex, and the amplified PCR products are analyzed using Illumina BeadChip (Shen et al., Mutat. Res., 573:70, 2005).

The molecular inversion probes (MIPs) are a method in which gap ligation is performed using a padlock probe, after which probes and genomic DNA not ligated to the DNA are removed using an exonuclease, and the padlock probe is linearized using uracil-N-glycosylase, followed by polymerase chain reaction using common primer nucleotide sequences included in the probes and hybridization to the GenFlex tag array (Affymetrix) to analyze various gene regions (Hardenbol et al., Nat. Biotechnol., 21:673, 2003).

However, these methods have problems in that, since portions of reaction products in a first tube are transferred and reacted in a second tube or several kinds of enzymes should be used, cross-contamination between samples can occur and experimental methods are complicated.

### SUMMARY OF THE INVENTION

Therefore, as a result of intensive efforts to solve the above-described problems and develop a method of detecting multiple targets using a single probe, the inventors of the present invention confirmed that, when amplifying multiple targets with primers including a tag sequence designed such that melting temperatures of hybridized products of amplification products and a detection probe are different from each other, hybridizing the targets with a single detection probe that binds to all of the tag sequences, and then analyzing melting curves, multiple targets can be detected with high sensitivity and high accuracy, thus completing the present invention.

### Technical Problem

Therefore, the present invention has been made in view of the above problems, and it is an object of the present invention to provide a method of detecting multiple targets.

It is another object of the present invention to provide a PCR composition for detecting multiple targets.

It is a further object of the present invention to provide a method of analyzing the expression levels of multiple target genes.

### Technical Solution

In accordance with an aspect of the present invention, the above and other objects can be accomplished by the provision of a method of detecting multiple targets, the method including: a) obtaining DNA from a sample containing multiple targets; b) amplifying multiple target nucleic acids using n primer sets capable of respectively amplifying n multiple target nucleic acids (wherein n is an integer of 2 to 20); c) hybridizing the n amplification products with a single detection probe capable of hybridizing with the n amplification products; and d) analyzing a melting curve of each of the n reaction products hybridized in process c) to determine the presence or absence of the target nucleic acids, wherein each of the n primer sets includes a forward primer and a reverse primer including a tag sequence, wherein the tag sequences are designed such that melting temperatures of the n hybridized reaction products are different from each other.

In accordance with another aspect of the present invention, there is provided a PCR composition for detecting multiple targets, the PCR composition including: i) n primer sets capable of respectively amplifying n targets; and ii) a detection probe capable of hybridizing with n amplification products amplified with the n primer sets (wherein n is an integer of 2 to 20), wherein each of the n primer sets consists of a forward primer and a reverse primer including a tag sequence, wherein the tag sequences are designed such that melting temperatures of the n hybridized reaction products are different from each other.

In accordance with a further aspect of the present invention, there is provided a method of analyzing expression levels of multiple target genes, including: a) obtaining a cDNA library from a sample containing multiple targets; b) amplifying a reference gene and target genes with a primer set capable of amplifying the reference gene and n primer sets capable of respectively amplifying n target genes (wherein n is an integer of 2 to 20); c) hybridizing the amplification products with a detection probe capable of hybridizing with all of the amplification product of the reference gene and the n amplification products; d) analyzing melting curves of reaction products hybridized in process c);

and e) comparing and analyzing Ct values at a melting temperature at which the reference gene and the target genes are simultaneously detectable and at a melting temperature at which only the target genes are detectable, wherein each of the n primer sets consists of a forward primer and a reverse primer including a tag sequence, wherein the tag sequences are designed such that melting temperatures of the n hybridized reaction products are different from each other.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view illustrating the concept of a method of detecting multiple targets according to the present invention;

FIG. 2 illustrates real-time polymerase chain reaction (PCR) conditions for detecting meningitis-related viruses and bacteria using a method of detecting multiple targets according to the present invention;

FIG. 3 illustrates the results of simultaneously detecting meningitis viruses and bacteria using a method of detecting multiple targets according to the present invention;

FIG. 4 is a view illustrating real-time PCR conditions for determining Tm values to analyze the expression levels of target genes with respect to a reference gene using a method of detecting multiple targets according to the present invention;

FIG. 5 illustrates the results of analyzing Ct values according to temperature for confirming expression levels of target genes with respect to a reference gene using a method of detecting multiple targets according to the present invention;

FIG. 6 is a view illustrating real-time PCR conditions for analyzing the expression levels of target genes with respect to a reference gene using a method of detecting multiple targets according to the present invention;

FIG. 7 illustrates the results of analyzing the expression level of a first target gene with respect to a reference gene using a method of detecting multiple targets according to the present invention; and

FIG. 8 illustrates the results of analyzing the expression level of a second target gene with respect to a reference gene using a method of detecting multiple targets according to the present invention.

### DETAILED DESCRIPTION AND EXEMPLARY EMBODIMENTS

Unless defined otherwise, all technical and scientific terms used herein have the same meanings as commonly understood by one of ordinary skill in the art to which the present invention pertains. In general, the nomenclature used herein and experimental methods described below are well known and commonly used in the art.

The present invention serves to confirm that, when amplifying targets with primer sets including tag sequences designed such that melting temperatures of hybridized reaction products of amplification products and a detection probe are different from each other, hybridizing the amplification products with a single probe that binds to all of the tag sequences, and then analyzing melting curves, multiple targets can be detected using a single probe.

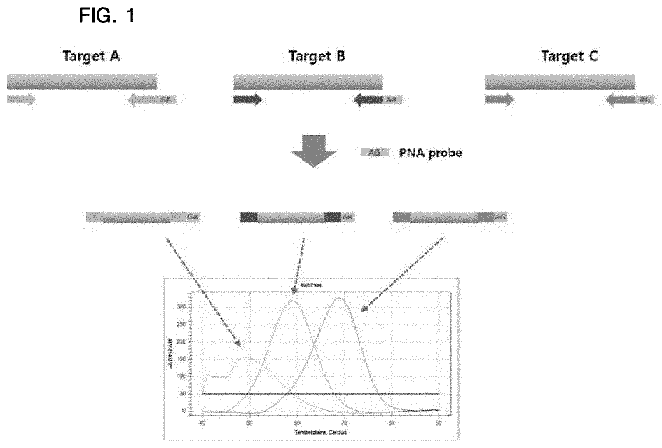

That is, in one embodiment of the present invention, when respective primer sets capable of amplifying 6 types of viruses causative of meningitis (HSV-1, HSV-2, VZV, CMV, EBV, and HHV-6) and 5 types of bacteria causative of meningitis (Streptococcus pneumoniae, Haemophilus influenza, Listeria monocytogenes, Group B Streptococcus, and Neisseria meningitides) were fused with different tag sequences for each virus and bacterium and then amplification products were produced and hybridized with a first detection probe, capable of binding to all of the tag sequences of 6 viruses, and a second detection probe, capable of binding to all of the tag sequences of 5 bacteria, after which melting curves were analyzed, it was confirmed that each virus and bacterium could be detected with high sensitivity (see FIGS. 1 to 3).

Therefore, an embodiment of the present invention relates to a method of detecting multiple targets, including:

a) obtaining DNA from a sample containing multiple targets;

b) amplifying multiple target nucleic acids using n primer sets capable of respectively amplifying n multiple target nucleic acids (wherein n is an integer of 2 to 20);

c) hybridizing the n amplification products with a single detection probe capable of hybridizing with the n amplification products; and

d) analyzing a melting curve of each of the n reaction products hybridized in process c) to determine the presence or absence of the target nucleic acids,

wherein each of the n primer sets includes

a forward primer and a reverse primer including a tag sequence, and

the tag sequences are designed such that melting temperatures of the n hybridized reaction products are different from each other.

The term “target” as used herein means all kinds of nucleic acids to be detected, and includes chromosomal nucleotide sequences derived from different species, subspecies, or variants, and chromosomal mutations within the same species. The target may include all types of DNA including genomic DNA, mitochondrial DNA, and viral DNA, or all types of RNA including mRNA, miRNA, ribosomal RNA, non-coding RNA, tRNA, and viral RNA, but the present invention is not limited thereto.

In the present invention, the target may be, but is not limited to, a mutant nucleotide sequence including a mutation in the nucleotide sequence, and the mutation may be selected from the group consisting of a single-nucleotide polymorphism (SNP), an insertion, a deletion, a point mutation, a fusion mutation, a translocation, an inversion, and loss of heterozygosity (LOH), but the present invention is not limited thereto.

In the present invention, the target may be, but is not limited to, a nucleic acid capable of detecting a specific bacterium or virus.

As used herein, the term “nucleoside” refers to a glycosylamine compound in which a nucleic acid base (nucleobase) is linked to a sugar moiety. A “nucleotide” means a nucleoside phosphate. As shown in Table 1, nucleotides may be represented using alphabetic letters (letter names) corresponding to nucleosides thereof. For example, A refers to adenosine (a nucleoside containing an adenine nucleobase), C refers to cytidine, G refers to guanosine, U refers to uridine, and T refers to thymidine (5-methyl uridine). W refers to A or T/U, and S refers to G or C. N denotes a random nucleoside, and dNTP means deoxyribonucleoside triphosphate. N may be any of A, C, G, or T/U.

TABLE 1

| Alphabetic | Nucleotide represented by |
|---|---|
| letter | alphabetic letter |
| G | G |
| A | A |
| T | T |
| C | C |
| U | U |
| R | G or A |
| Y | T/U or C |
| M | A or C |
| K | G or T/U |
| S | G or C |
| W | A or T/U |
| H | A or C or T/U |
| B | G or T/U or C |
| V | G or C or A |
| D | G or A or T/U |
| N | G or A or T/U or C |

As used herein, the term “oligonucleotide” refers to an oligomer of nucleotides. As used herein, the term “nucleic acid” refers to a polymer of nucleotides. As used herein, the term “sequence” refers to the nucleotide sequence of an oligonucleotide or nucleic acid. Throughout the specification, whenever an oligonucleotide or nucleic acid is represented by a sequence of letters, the nucleotides are in an 5′→order from left to right. Oligonucleotides or nucleic acids may be DNA, RNA, or analogues thereof (e.g., phosphorothioate analogues). Oligonucleotides or nucleic acids may also include modified bases and/or backbones (e.g., modified phosphate linkages or modified sugar moieties). Non-limiting examples of synthetic backbones that impart stability and/or other advantages to nucleic acids may include phosphorothioate linkages, peptide nucleic acids, locked nucleic acids, xylose nucleic acids, or analogues thereof.

As used herein, the term “nucleic acid” refers to a nucleotide polymer and includes known analogues of natural nucleotides that can act in a manner similar to naturally occurring nucleotides (e.g., hybridization) unless otherwise defined.

The term “nucleic acid” includes any form of DNA or RNA including, for example, genomic DNA, complementary DNA (cDNA), which is a DNA representation of mRNA, usually obtained by reverse transcription or amplification of messenger RNA (mRNA), DNA molecules produced synthetically or by amplification, and mRNA.

The term “nucleic acid” encompasses double- or triple-stranded nucleic acids as well as single-stranded molecules. In double- or triple-stranded nucleic acids, nucleic acid strands need not be coextensive (i.e., double-stranded nucleic acids need not be double-stranded along the full length of both strands).

The term “nucleic acid” also includes any chemical modification thereof, such as by methylation and/or by capping. Nucleic acid modifications may include addition of chemical groups imparting an additional charge, polarizability, hydrogen bonding, electrostatic interaction, or other functionality to individual nucleic acid bases or to a nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, backbone modifications, and unusual base-pairing combinations such as the isobases isocytidine and isoguanidine.

Nucleic acid(s) may be derived through a complete chemical synthesis process, such as solid-phase-mediated chemical synthesis, from a biological source such as isolation from any species that produces a nucleic acid, from processes that involve handling of nucleic acids by molecular biological tools, such as DNA replication, PCR amplification, and reverse transcription, or from a combination of these processes.

As used herein, the term “complementary” refers to the capacity for accurate pairing between two nucleotides. That is, if a nucleotide at a given position of a nucleic acid is capable of forming a hydrogen bond with a nucleotide of another nucleic acid, two nucleic acids are considered to be complementary to each other at that position. Complementarity between two single-stranded nucleic acid molecules may be “partial,” in which only some of the nucleotides bind, or may be complete when total complementarity exists between single-stranded molecules. The degree of complementarity between nucleic acid strands has considerable effects on the efficiency and strength of hybridization between nucleic acid strands.

As used herein, the term “primer” refers to a short linear oligonucleotide that hybridizes to a target nucleic acid sequence (e.g., a DNA template to be amplified) for programming a nucleic acid synthesis reaction. The primer may be an RNA oligonucleotide, a DNA oligonucleotide, or a chimeric sequence. Primers may include natural, synthetic, or modified nucleotides. Both the upper and lower limits of the length of the primer are experimentally determined. The lower limit on primer length is the minimum length that is required to form a stable duplex upon hybridization with a target nucleic acid under nucleic acid amplification reaction conditions. Very short primers (usually less than 3 nucleotides long) do not form thermodynamically stable duplexes with target nucleic acids under such hybridization conditions. The upper limit is generally determined by the possibility of forming a duplex in a region other than the predetermined nucleic acid sequence. Generally, the length of suitable primers may range from about 3 nucleotides to about 50 nucleotides in length.

As used herein, the term “probe” refers to a nucleic acid capable of binding to a target nucleic acid having a complementary sequence through one or more types of chemical bonds, generally through complementary base pairing, usually through hydrogen bond formation, thus forming a duplex structure. Probes bind or hybridize to “probe-binding sites.” Specifically, once a probe hybridizes to a target complementary to the probe, the probe may be labeled with a detectable label to facilitate probe detection. Alternatively, however, the probe may be unlabeled, but may be detectable by specific binding with a labeled ligand, either directly or indirectly. Probes may vary considerably in size. Probes generally have a length of at least 7 to 18 nucleotides. Other probes have a length of at least 20, 30, or 40 nucleotides. Still other probes are somewhat longer, and have a length of at least 50, 60, 70, 80, or 90 nucleotides. Yet other probes are much longer, and have a length of at least 100, 150, 200 or more nucleotides. Probes may also have a length within any range limited by any of the above values (e.g., 15-20 nucleotides in length).

As used herein, the term “hybridization” refers to the formation of double-stranded nucleic acids by hydrogen bonding between single-stranded nucleic acids having complementary nucleotide sequences, and is used interchangeably with the term “annealing.” In a slightly broader sense, hybridization includes not only the case where nucleotide sequences between two single strands are completely complementary (perfect match), but also exceptionally includes the case where some nucleotide sequences are not complementary (mismatch).

As used herein, the term “sample” refers to a composition that contains or is assumed to contain a target and is to be analyzed, and may be a sample collected from any one or more selected from liquid, soil, air, food, waste, substances derived from humans, animal intestines, and animal and plant tissues, but the present invention is not limited thereto. In this regard, the liquid may be water, blood, urine, tears, sweat, saliva, lymph, cerebrospinal fluid, and the like, the water includes river water, seawater, lake water, rain water, and the like, the waste includes sewage, waste water, and the like, and the animal and plant tissues include those of humans. In addition, the animal and plant tissues include tissues such as mucous membranes, skin, cortices, hair, scales, eyes, tongues, cheeks, hooves, beaks, snouts, feet, hands, mouths, nipples, ears, and noses.

Preferably, the sample of the present invention may be a biological sample to be analyzed using the method of the present invention. More preferably, the sample may be a sample mixed with a virus species or a sample of an individual (e.g., a human, a mammal, and fish) infected with the virus, and a biological sample derived from a plant, an animal, a human, fungus, a bacterium, and a virus may be analyzed. When a sample of mammalian or human origin is analyzed, the sample may be derived from a particular tissue or organ. Representative examples of tissues include connective, skin, muscle or nerve tissues. Representative examples of organs include the eyes, brain, lungs, liver, spleen, bone marrow, thymus, heart, lymph, blood, bone, cartilage, pancreas, kidneys, gallbladder, stomach, small intestine, testes, ovaries, uterus, rectum, nervous system, glands, and internal blood vessels. The biological sample to be analyzed includes any cell, tissue, or fluid from a biological origin, or any other medium that can be analyzed according to the present invention, and includes samples from humans, animals, or foods prepared for consumption by humans or animals. In addition, the biological sample to be analyzed includes body fluid samples, and examples thereof include, but are not limited to, blood, serum, plasma, lymph, breast milk, urine, feces, ocular fluid, saliva, semen, brain extracts (e.g., ground brain sample), spinal fluid, appendix, spleen, and tonsil tissue extracts.

In the present invention, the amplification may be performed through any kind of polymerase chain reaction (PCR), but preferably, asymmetric PCR may be used.

In the present invention, the tag sequences may have a length of 5 bp to 50 bp.

In the present invention, the tag sequences may have a GC ratio of 20% to 80%.

In the present invention, the melting temperature by the tag sequence may be adjusted according to the composition or length of the tag sequence.

In the present invention, the tag sequence may be complementary to a probe sequence or a sequence containing the probe sequence.

In the present invention, the difference between melting temperatures is not particularly limited, as long as it is distinguishable on an analysis graph, but may preferably range from 2° C. to 40° C., more preferably 5° C. to 30° C., and most preferably 8° C. to 20° C.

In the present invention, in process b), p primer sets capable of respectively detecting p targets may further be included (wherein p is an integer of 1 to 20), and in process c), a detection probe capable of hybridizing to all of the p amplification products may further be included.

In the present invention, the detection probe may be an oligonucleotide, a peptide nucleic acid (PNA), or a locked nucleic acid (LNA), and may have a reporter and a quencher attached to opposite ends thereof.

In the present invention, a peptide nucleic acid (PNA) is a substance that recognizes genes, like a locked nucleic acid (LNA) or a morpholino nucleic acid (MNA), is artificially synthesized, and has a backbone consisting of polyamide. PNA is excellent in affinity and selectivity and has high stability for nucleolytic enzymes, and thus is not cleaved by existing restriction enzymes. In addition, PNA has excellent thermal/chemical properties and stability, and thus storage thereof is easy and PNA is not easily decomposed. In addition, PNA-DNA binding affinity is much higher than DNA-DNA binding affinity, and thus there is a difference in melting temperature (Tm) of about 10° C. to 15° C. even in the presence of a single-nucleotide mismatch. Using this difference in binding affinity, single-nucleotide polymorphisms (SNPs) and insertion/deletion (InDel) nucleotide changes can be detected.

The Tm value also changes depending on the difference between the nucleic acid of a PNA probe and DNA that binds complementarily thereto, and thus the development of application techniques using the same is easy. The PNA probe is analyzed using a hybridization reaction different from the hydrolysis reaction of a TaqMan probe, and probes having functions similar to that of the PNA probe include molecular beacon probes and scorpion probes.

In the present invention, the PNA probe is not limited, but may have a reporter or quencher bound thereto. The PNA probe of the present invention, including a reporter and a quencher generates a fluorescent signal after hybridizing to the target nucleic acid, and as the temperature increases, the PNA probe is rapidly melted with the target nucleic acid at a suitable melting temperature thereof, and thus the fluorescent signal is quenched. Through analysis of a high-resolution melting curve obtained from the fluorescent signal according to temperature changes, the presence or absence of a target nucleic acid may be detected.

A fluorescent material may bind to the probe of the present invention including, at opposite ends thereof, a reporter and a quencher capable of quenching the fluorescence of the reporter, and may include an intercalating fluorescent material. The reporter may be one or more selected from the group consisting of 6-carboxyfluorescein (FAM), HEX, Texas Red, JOE, TAMRA, CY5, CY3, and Alexa680, and as the quencher, 6-carboxytetramethyl-rhodamine (TAMRA), BHQ1, BHQ2, or Dabcyl is preferably used, but the present invention is not limited thereto. The intercalating fluorescent material may be selected from a group consisting of acridine homodimer and derivatives thereof, acridine orange and derivatives thereof, 7-aminoactinomycin D (7-AAD) and derivatives thereof, actinomycin D and derivatives thereof, 9-amino-6-chloro-2-methoxyacridine (ACMA) and derivatives thereof, DAPI and derivatives thereof, dihydroethidium and derivatives thereof, ethidium bromide and derivatives thereof, ethidium homodimer-1 (EthD-1) and derivatives thereof, ethidium homodimer-2 (EthD-2) and derivatives thereof, ethidium monoazide and derivatives thereof, hexidium iodide and derivatives thereof, bisbenzimide (Hoechst 33258) and derivatives thereof, Hoechst 33342 and derivatives thereof, Hoechst 34580 and derivatives thereof, hydroxystilbamidine and derivatives thereof, LDS 751 and derivatives thereof, propidium iodide (PI) and derivatives thereof, and Cy-dyes derivatives.

In the present invention, fluorescence melting curve analysis (FMCA) is used as a method of analyzing a hybridization reaction, and is performed by classifying, based on melting temperatures, differences in binding strength between products produced after completion of PCR and the introduced probe. Unlike other SNP detection probes, design of the probe is very simple such that the probe is produced using 11 to 18 mer nucleotide sequences containing SNPs. Thus, to design a probe with a desired melting temperature, the Tm value may be adjusted according to the length of the PNA probe, and even in the case of PNA probes of the same length, the Tm value may be adjusted by changing the probes. Since PNA has higher binding strength than DNA and has a high basic Tm value, it is possible to design PNA with a shorter length than DNA, so that even SNPs closely adjacent thereto can be detected. In a conventional HRM method, a difference in Tm value is as low as about 0.5° C., and thus an additional analytic program or a minute change in temperature is required, and it is difficult to perform analysis when two or more SNPs appear. In contrast, the PNA probe is not influenced by SNPs other than the probe sequence, and enables rapid and accurate analysis.

In an exemplary embodiment of the present invention, detection of the fused amplification product is carried out through real-time PCR, and in this regard, may be performed by obtaining only an amplification curve according to amplification of the fused amplification product to measure a cycle threshold (Ct) value, obtaining only a melting curve after polymerase chain reaction to measure a melting peak by the probe, or obtaining both the amplification curve and the melting curve and taking the two results together, but the present invention is not limited thereto.

Because the fused amplification product is amplified early due to the presence of a target nucleic acid in a sample, the amount of signal generated by the detection probe increases early, and thus the number of cycles required to reach the threshold decreases so that a low Ct value is measured, which may be used to identify the presence or absence of the target nucleic acid. In addition, melting curve analysis is generally performed after a nucleic acid amplification process of real-time polymerase chain reaction, and a signal pattern is measured while the temperature of a sample is raised at a rate of 0.3-1° C. per 1-10 seconds up to a high temperature (approximately 75° C. to 95° C.) after being lowered to a low temperature (approximately 25° C. to 55° C.) or is lowered at a rate of 0.3-1° C. every 1-10 seconds to the low temperature after being raised to the high temperature. When the fused amplification product is amplified, a change in signal pattern appears around the melting temperature (Tm) of a probe bound to the fused amplification product through the melting curve analysis, and may be analyzed using a melting peak to identify the fused amplification product.

The present invention also relates to a PCR composition for detecting multiple targets, including:

i) n primer sets capable of respectively amplifying n targets; and

ii) a detection probe capable of hybridizing with n amplification products amplified with the n primer sets (wherein n is an integer of 2 to 20),

wherein each of the n primer sets consists of a forward primer and a reverse primer including a tag sequence, and

the tag sequences are designed such that melting temperatures of n hybridized reaction products are different from each other.

In the present invention, the PCR composition may further include p primer sets capable of respectively detecting p targets (wherein p is an integer of 1 to 20), and may further include a detection probe capable of hybridizing with all of the p amplification products.

The present invention also relates to a kit for detecting multiple targets.

In the present invention, the kit may optionally include reagents needed for target nucleic acid amplification reaction (e.g., polymerase chain reaction), such as a buffer, a DNA polymerase, a DNA polymerase cofactor, and deoxyribonucleotide-5-triphosphates (dNTPs). Optionally, the kit of the present invention may also include various oligonucleotide molecules, reverse transcriptase, various buffers and reagents, and antibodies for inhibiting the activity of DNA polymerases. In addition, the optimum amount of a reagent used in a specific reaction of the kit may be readily determined by one of ordinary skill in the art. Typically, the equipment of the present invention may be manufactured in a separate package or compartment including the aforementioned components.

In one embodiment, the kit may include a partitioned carrier means for containing a sample, a vessel including a reagent, a vessel including a surrogate target and primers, and a vessel including a probe for detecting the amplification products.

The carrier means is suitable for containing one or more vessels such as bottles and tubes, and each container includes independent components used in the method of the present invention. In the present specification, one of ordinary skill in the art can readily dispense agents required in the vessels.

Meanwhile, in the present invention, it was expected that the expression levels of target genes with respect to a reference gene could be compared and analyzed using the detection method.

In the present invention, to confirm whether the expression levels of target genes with respect to a reference gene can be analyzed, the reference gene and the target genes were amplified with respective primer sets each including a tag sequence, and then a melting curve was analyzed with a single detection probe to determine a melting temperature at which all of the reference gene and the target genes are detectable and a melting temperature at which only the target genes are detectable, and Ct values at the respective melting temperatures were compared and analyzed.

That is, in one embodiment of the present invention, β-actin was set as a reference gene, PD-1 and PD-L1 were set as target genes, cDNA was prepared from mRNA of cell lines Hcc827, MDA, and MRC5, each gene was amplified with primers including a tag sequence, a detection probe capable of binding to the tag sequences was hybridized with the amplification products, and then a melting curve was analyzed, and from the analysis results, it was confirmed that a temperature at which β-actin and PD-1/PD-L1 are simultaneously detectable was ° C., and a temperature at which only PD-1/PD-L1 is detectable was 58° C.

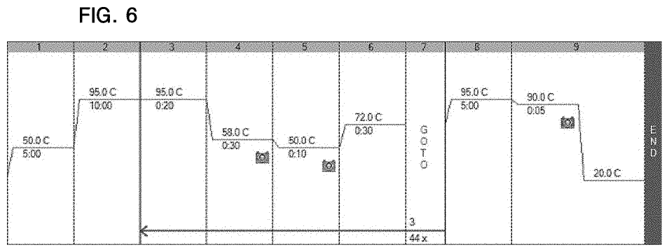

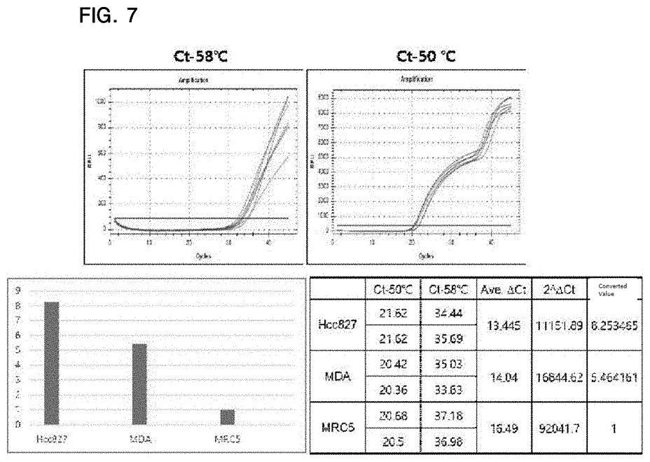

Thereafter, a Ct value at each temperature was measured and, as a result of comparing and analyzing the difference therebetween, it was confirmed that, based on the expression of β-actin and PD-1/PD-L1 in MRC5 cells in which PD-1/PD-L1 is normally expressed, PD-1/PD-L1 were expressed in Hcc82 eight times/18 times, respectively, more than β-actin, and expressed in MDA five times/55 times more than β-actin (see FIGS. 6 and 7).

Therefore, another embodiment of the present invention relates to a method of analyzing expression levels of multiple target genes, including:

a) obtaining a cDNA library from a sample containing multiple targets;

b) amplifying a reference gene and target genes with a primer set capable of amplifying the reference gene and n primer sets capable of respectively amplifying n target genes (wherein n is an integer of 2 to 20);

c) hybridizing the amplification products with a detection probe capable of hybridizing with all of the amplification product of the reference gene and the n amplification products;

d) analyzing melting curves of reaction products hybridized in process c); and

e) comparing and analyzing Ct values at a melting temperature at which the reference gene and the target genes are simultaneously detectable and at a melting temperature at which only the target genes are detectable,

wherein each of the n primer sets consists of a forward primer and a reverse primer including a tag sequence, and

the tag sequences are designed such that melting temperatures of n hybridized reaction products are different from each other.

In the present invention, the comparison and analysis of the Ct values of process e) may be performed by:

i) obtaining a difference between a Ct value at a melting temperature at which the reference gene and the target genes are simultaneously detectable and a Ct value at a melting temperature at which only the target genes were detectable;

ii) converting the difference between the Ct values using Equation 1 below; and

converted value=2\hat{ }(Ct value at melting temperature at which only target genes are detectable−Ct value at melting temperature at which reference gene and target genes are simultaneously detectable) of control/2\hat{ }(Ct value at melting temperature at which only target genes are detectable−Ct value at melting temperature at which reference gene and target genes are simultaneously detectable) of experimental group Equation 1:

iii) confirming expression levels compared to the reference gene through the converted value.

In the present invention, the cDNA library may be obtained from a sample using various known methods, and preferably, the cDNA library may be obtained from extracted mRNA through reverse transcriptase PCR (RT-PCR).

In the present invention, for diagnosis and treatment of cancer, the experimental group or target gene may be any one or more genes selected from the group consisting of PD-1, PD-L1, CTL4, LAG3, TIM3, BILA, TIGIT, VISTA, KIR, A2AR, B7-H3, B7-H4, CD277, and IDO, or may be any one or more selected from the group consisting of miR-17, miR-18a, miR-20a, miR-21, miR-27a, and miR-155, but the present invention is not limited thereto.

In the present invention, the control or reference gene may be any one or more house-keeping genes selected from the group consisting of β-actin, α-tubulin, and GAPDH, but the present invention is not limited thereto.

Hereinafter, the present invention will be described in further detail with reference to the following examples. It will be obvious to those of ordinary skill in the art that these examples are provided for illustrative purposes only and should not be construed as limiting the scope of the present invention.

### EXAMPLES

### Example 1: Detection of 6 Viruses and 5 Bacteria Causative of Meningitis

To detect 6 viruses causative of meningitis (HSV-1, HSV-2, VZV, CMV, EBV, and HHV-6), and 5 bacteria causative of meningitis (Streptococcus pneumoniae, Haemophilus influenza, Listeria monocytogenes, Group B Streptococcus, Neisseria meningitides), forward primers, reverse primers each including a tag sequence, and bifunctional PNA fluorescent probes were produced (see Tables 2 and 3).

TABLE 2

| SEQ | | | |
|---|---|---|---|
| ID | | | |
| NO: | Name | Sequence (5′-3′) | Target |
| 1 | V1-F | GCTGTTCTCGTTCCTCACTGCC | HSV-1 |
| 2 | V1-R | TGAAAATGCGAGTGTCCATACCCTACCCGCGTTCGGAC | |
| 3 | V2-F | CGCCAAATACGCCTTAGCAGAC | HSV-2 |
| 4 | V2-R | TGAAAATGGAAGTGTCAGGTTCTTCCCGCGAAATCG | |
| 5 | V3-F | CCTTCAATTGCTTGGCGGACTCGG | VZV |
| 6 | V3-R | TGATAATGCAAGTCTCACAAGATGAGCGAGTGTACCGATG | |
| 7 | V4-F | GCTGTAACTGTGGTTTCCATGACG | CMV |
| 8 | V4-R | TGAAAATGCAAGTGTCCGTGTGGCTTACCTGCTGCC | |
| 9 | V5-F | AGCGGGGTATGAGCTTTCCTGTTAC | EBV |
| 10 | V5-R | TCAAAATGCAAGTGTCCAGTCGGGCGAAATCTGTGTACC | |
| 11 | V6-F | GATATCGGATCGCAACAAGACCTCG | HHV-6 |
| 12 | V6-R | TGAAGATGGAAGTGTCTCCGTTGCGTAATATGTCAAGGATGC | |
| 13 | B1-F | GGTCAATTCCTGTCGCAGTACC | Streptococcus |
| 14 | B1-R | CATGTGCCTACACCTGGTCCAAACAGCCTTAGGTCTTATGG | pneumoniae |
| 15 | B2-F | GTACGCTAACACTGCACGACG | Haemophilus |
| 16 | B2-R | CATGTGCATACACCTGGTAACACTGATGAACGTGGTACACCAG | influenzae |
| 17 | B3-F | GTTGACCGCAAATAGAGCCAAGC | Listeria |
| 18 | B3-R | CATGTGCCTACACGAGGTATTAGCGAGAACGGGACCATCATG | monocytogenes |
| 19 | B4-F | CAGCAACAACGATTGTTTCGCC | Group B |
| 20 | B4-R | CATGTCCATACACCTGCTTCCTCTTTAGCTGCTGGAAC | Streptococcus |
| 21 | B5-F | GCACACTTAGGTGATTTACCTGCAT | Neisseria |
| 22 | B5-R | CATATCCCTACACCTGCCACCCGTGTGGATCATAATAGA | meningitidis |

Underlined letters; 5′-tagging sequence of reverse primer

TABLE 3
Probe sequences

| SEQ | | | |
|---|---|---|---|
| ID | | Sequence | |
| NO: | Name | (5′-3′) | Fluor. |
| 23 | Detect | CAT GTG CCT | FAM |
| | P1 | ACA CCT G | |
| 24 | Detect | TGA AAA TGC | TxR |
| | P2 | AAG TGT C | |

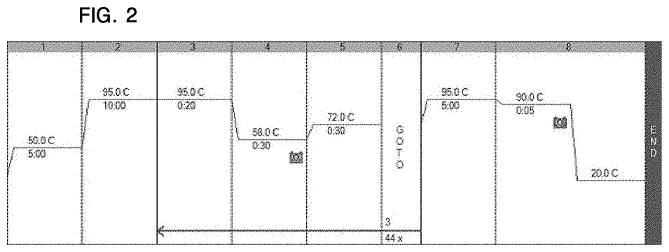

A real-time polymerase chain reaction experiment was conducted using asymmetric PCR to produce single-stranded target nucleic acids. Asymmetric PCR conditions are as follows: 2X SeaSunBio Real-Time FMCA™ buffer (SeaSunBio, Korea); 2.5 mM MgCl₂; 200 μM dNTPs; 1.0 U Taq polymerase; 0.05 μM forward primer (see Table 2); and 0.5 μM reverse primer (see Table 2) (asymmetric PCR), and 0.5 μl of the fluorescent PNA probes (see Table 3) were added to a final volume of 20 it to conduct real-time PCR analysis and melting curve analysis and analysis conditions are shown in FIG. 2.

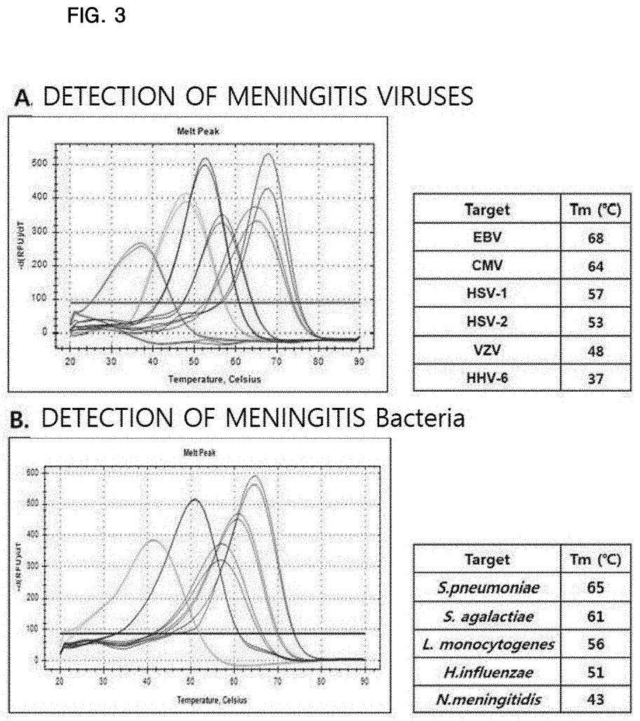

As a result, as illustrated in FIG. 3, it was confirmed that 5 bacteria and 6 viruses, which are causative of meningitis, were detectable.

The origins of each virus and each bacterium are shown in Table 4 below.

TABLE 4
Origins of viruses and bacteria

| No. | Name | Origin |
|---|---|---|
| 1 | Streptococcus pneumoniae | ATCC27336 |
| 2 | Neisseria meningitidis | ATCC13100 |
| 3 | Haemophilus influenzae | ATCC19418 |
| 4 | Listeria monocytogenes | ATCC15313 |
| 5 | Streptococcus agalactiae | ATCC14364 |
| 6 | Human herpesvirus 1 | KBPV-VR-52 |
| 7 | Human herpesvirus 2 | KBPV-VR-53 |
| 8 | Varicella zoster virus | AMX VZV Plasma Pnl, Acrometrix |
| 9 | Epstein-Barr virus | Acromatrix panel |
| 10 | Human cytomegalovirus | KBPV-VR-7 |
| 11 | Human herpesvirus 6 | HHV-6 Virus 1st WHo International |
| | | standard |

### Example 2: Gene Expression Level Analysis

2-1. Determination of Melting Temperatures of Control and Experimental Groups

To compare gene expression, standard cell lines Hcc827, MDA, and MRC5 were selected (EA. Mittendorf et al., 2014, RHJ Janse et al., 2018, H Soliman et al., 2014), and cDNA was synthesized from RNA extracted from the corresponding cell lines using a SuperiorScrip III Reverse Transcriptase (Enzynomics, RT006) kit. Conditions for cDNA synthesis are as follows: 5× Fist-Strand buffer, 200 units of SuperiorScriptIII Reverse Transcriptase, 0.5 mM dNTP Mixture, 10 mM DTT, 4 μM oligo dT, 20 units of RNase inhibitor were added to a total volume of 20 μl to cause a reaction to occur at 37° C. for 5 minutes, 50° C. for 1 hour, and 70° C. for 15 minutes.

To analyze gene expression, primers of a reference gene and target genes were prepared as shown in Table 5. PCR was performed in a CFX96™ Real-Time system (BIO-RAD, U.S.) using the bifunctional PNA fluorescent probes produced according to Example 1.

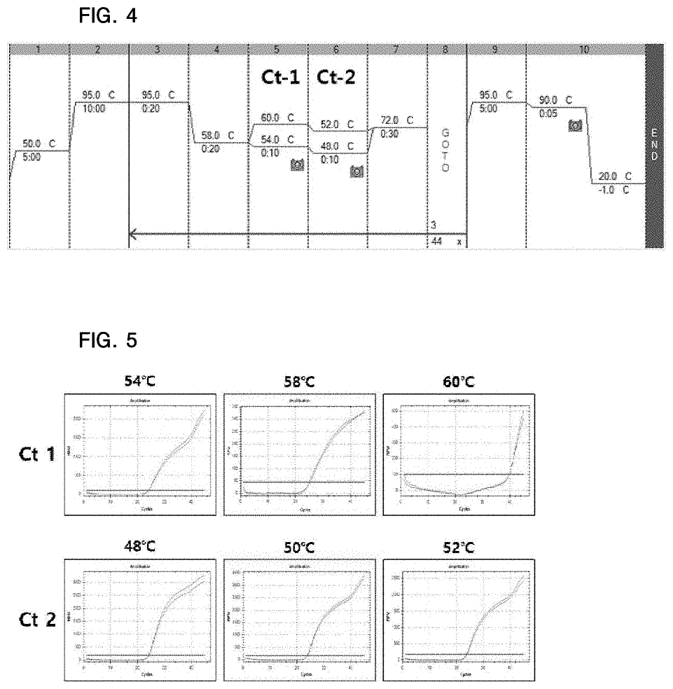

A real-time polymerase chain reaction experiment was conducted using asymmetric PCR to produce single-stranded target nucleic acids. Asymmetric PCR conditions are as follows: 2× SeaSunBio Real-Time FMCA™ buffer (SeaSunBio, Korea); 2.5 mM MgCl₂; 200 μM dNTPs; 1.0 U Taq polymerase; 0.05 μM forward primer (see Table 2); and 0.5 μM reverse primer (see Table 2) (asymmetric PCR), and 0.5 μl of the fluorescent PNA probes (see Table 3) were added to a final volume of 20 it to conduct real-time PCR analysis and melting curve analysis and analysis conditions are illustrated in FIG. 4.

To determine annealing temperatures suitable for the analysis of Ct values of β-actin and PD-1/PD-L1, detection conditions of PD-1/PD-L1 were set to 54° C. to 60° C. and conditions for β-actin and PD-1/PD-L1 to be simultaneously detectable were set to 48° C. to 52° C. and analysis was conducted, from which it was confirmed that 50° C. and 58° C. are the most suitable temperatures for analysis (see FIG. 5).

TABLE 5
Primer sequences

| SEQ | | | |
|---|---|---|---|
| ID | | | |
| NO: | Name | Sequence (5′-3′) | Target |
| 25 | β-actin-F | GCACTCTTCCAGCCTTCC | β-actin |
| 26 | β-actin-R | TGAAAATGGAAGTGTCAGCACTGTGTTGGCGTACAG | β-actin |
| 27 | PD-1-F | CAGAGCTCAGGGTGACAGAGAG | PD-1 |
| 28 | PD-1-R | TGAAAATGCAAGTCTCCCACGACACCAACCACCAGG | PD-1 |
| 29 | PD-L1-F | TGCTGAACGCATTTACTGTCACGG | PD-L1 |
| 30 | PD-L1-R | TGAAAATGCAAGTCTCACCATAGCTGATCATGCAGC | PD-L1 |
| | | GGTA | |

Underlined letters; 5′-tagging sequence of reverse primer

2-2. Analysis of Expression Levels of Control and Experimental Groups

To measure Ct values at melting temperatures of 50° C. and 58° C. determined using the method of Example 2-1, real-time PCR was performed using the materials as in Example 2-1 under conditions shown in FIG. 6, and then the gene expression level was analyzed using Equation below:

2\hat{ }(Ct58−Ct50) of reference gene/2\hat{ }(Ct58−Ct50) of target gene Equation for gene expression level analysis:

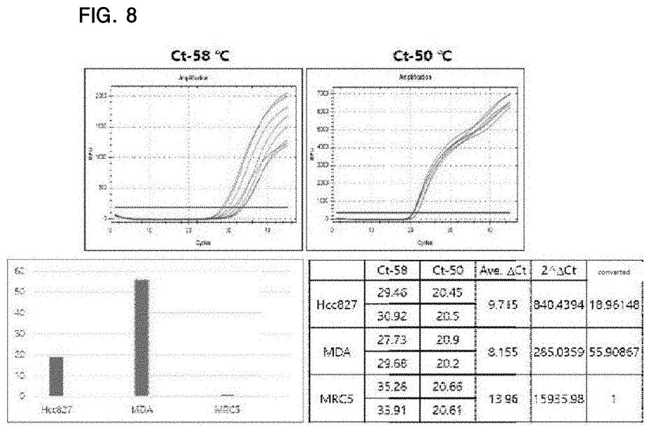

From the results, as illustrated in FIGS. 7 and 8, it was confirmed that the PD-1 and PD-L1 genes showed an expression level difference in HCC-827 and MDAMB-231 cells, compared to an MRC-5 cell line as a control.

Origins of the cell lines are shown in Table 6.

TABLE 6
Origins of Cell lines

| No. | Cell line name | Origin |
|---|---|---|
| 1 | MRC-5 | KCLB10171 |
| 2 | HCC-827 | KCLB70827 |
| 3 | MDAMB-231 | ATCC HTB-26 |

While specific embodiments of the present invention have been described in detail, it will be obvious to those of ordinary skill in the art that such detailed descriptions are provided for illustrative purposes only and are not intended to limit the scope of the present invention. Thus, the substantial scope of the present invention should be defined by the appended claims and equivalents thereto.

### INDUSTRIAL APPLICABILITY

A method of detecting multiple targets according to the present invention enables the detection of multiple targets using a single probe, and thus is useful for detecting multiple targets because false positives are reduced and multiple targets are detectable with high sensitivity and at a rapid rate.

1. A method of detecting multiple targets, the method comprising the steps of:
a) obtaining DNA from a sample containing multiple targets;
b) amplifying multiple target nucleic acids using n primer sets capable of respectively amplifying n multiple target nucleic acids (wherein n is an integer of 2 to 20);
c) hybridizing the n amplification products with a single detection probe capable of hybridizing with the n amplification products; and
d) analyzing a melting curve of each of the n reaction products hybridized in process c) to determine the presence or absence of the target nucleic acids,
wherein each of the n primer sets comprises
a forward primer and a reverse primer comprising a tag sequence,
wherein the tag sequences are designed such that melting temperatures of the n hybridized reaction products are different from each other.

2. The method according to claim 1, wherein the melting temperature difference ranges from 2° C. to 40° C.

3. The method according to claim 1, wherein in step b), p primer sets capable of respectively detecting p targets (wherein p is an integer of 1 to 20) are further included, and in step c), a detection probe capable of hybridizing with all of the p amplification products is further included.

4. The method according to claim 1, wherein the detection probe is an oligonucleotide, a peptide nucleic acid (PNA), or a locked nucleic acid (LNA), and has, on opposite ends thereof, a reporter and a quencher that are bound thereto.

5. The method according to claim 4, wherein the reporter comprises one or more selected from the group consisting of 6-carboxyfluorescein (FAM), Texas Red, 2′,4′,5′,7′,-tetrachloro-6-carboxy-4,7-dichlorofluorescein (HEX), and CY5.

6. The method according to claim 4, wherein the quencher comprises one or more selected from the group consisting of 6-carboxytetramethyl-rhodamine (TAMRA), BHQ1, BHQ2, and Dabcyl.

7. The method according to claim 1, wherein the analyzing of the melting curve is performed by fluorescence melting curve analysis (FMCA).

8. The method according to claim 1, wherein the amplifying is performed by real-time polymerase chain reaction (PCR).

9. The method according to claim 1, wherein the sample is selected from water, soil, waste, foods, substances derived from humans, animal intestines, and animal and plant tissues.

10. A PCR composition for detecting multiple targets, the PCR composition comprising:
i) n primer sets capable of respectively amplifying n targets; and
ii) a detection probe capable of hybridizing with n amplification products amplified with the n primer sets (wherein n is an integer of 2 to 20),
wherein each of the n primer sets consists of a forward primer and a reverse primer comprising a tag sequence,
wherein the tag sequences are designed such that melting temperatures of the n hybridized reaction products are different from each other.

11. The PCR composition according to claim 10, further comprising p primer sets capable of respectively detecting p targets (wherein p is an integer of 1 to 20), and further comprising a detection probe capable of hybridizing with all of the p amplification products.

12. A method of analyzing expression levels of multiple target genes, the method comprising the steps of:
a) obtaining a cDNA library from a sample containing multiple targets;
b) amplifying a reference gene and target genes with a primer set capable of amplifying the reference gene and n primer sets capable of respectively amplifying n target genes (wherein n is an integer of 2 to 20);
c) hybridizing the amplification products with a detection probe capable of hybridizing with all of the amplification product of the reference gene and the n amplification products;
d) analyzing melting curves of reaction products hybridized in process c); and
e) comparing and analyzing Ct values at a melting temperature at which the reference gene and the target genes are simultaneously detectable and at a melting temperature at which only the target genes are detectable,
wherein each of the n primer sets consists of a forward primer and a reverse primer comprising a tag sequence,
wherein the tag sequences are designed such that melting temperatures of the n hybridized reaction products are different from each other.

13. The method according to claim 12, wherein the comparing and analyzing of the Ct values of step e) is performed by:
i) obtaining a difference between a Ct value at a melting temperature at which the reference gene and the target genes are simultaneously detectable and a Ct value at a melting temperature at which only the target genes were detectable;
ii) converting the difference between the Ct values using Equation 1 below; and
converted value=2\hat{ }(Ct value at melting temperature at which only target genes are detectable−Ct value at melting temperature at which reference gene and target genes are simultaneously detectable) of control/2\hat{ }(Ct value at melting temperature at which only target genes are detectable−Ct value at melting temperature at which reference gene and target genes are simultaneously detectable) of experimental group Equation 1:
iii) confirming expression levels compared to the reference gene through the converted value.
